(19) United States
(12) Patent Application Publication
(10) Pub. No.: US 2006/0051223 A1
(43) Pub. Date: Mar. 9, 2006

(54) Hydrotransformer

(76) Inventors: Alexander Mark, Zell (DE); Rudolf Schaffer, Marktheidenfeld (DE)

Correspondence Address: Martin A Farber, Suite 473, 866 United Nations Plaza, New York, NY, 10017, US

(21) Appl. No.: 10/509,990

(22) PCT Filed: Apr. 10, 2003
(86) PCT No.: PCT/EP03/03710

§ 371 Date: Sep. 30, 2004

(30) Foreign Application Priority Data
Apr. 17, 2002 (DE) 102-16-951.9

(51) Int. Cl. F04B 27/08; A61M 1/00

(52) U.S. Cl. 417/437; 417/269

(57) ABSTRACT

The invention is based on a hydraulic transformer having a housing and having an expeller part in which a plurality of expellers which bound a number of expeller spaces with variable volumes are guided, a cam part on which the expellers are supported, and a valving surface which has three kidney-shaped control slots, one of which is connected to a supply port, one to a working port and one to a reservoir tank port. The objective is to provide a hydraulic transformer in which complex setting of the transmission ratio is avoided. The aimed-at objective is achieved according to the invention in that the valving surface can be driven in rotation by means of a drive, and in that of the two components comprising the expeller part and cam element one component is essentially arranged fixedly with respect to the housing and the other component can move freely in terms of two rotational or translatory degrees of freedom within a limited range.

The invention is based on a hydraulic transformer which has, according to the preamble of patent claim 1, a housing and an expeller part in which a plurality of expellers which bound expeller spaces with variable volumes are guided, a cam part on which the expellers are supported, and control means, in particular a control cam with three kidney-shaped control slots via which the expeller spaces can be successively connected to a supply port, to a working port and to a reservoir tank port.

A hydraulic transformer is a hydraulic machine in which a hydraulic motor and a hydraulic pump are mechanically connected to one another and the hydraulic motor drives the hydraulic pump. At least the swept volume of the hydraulic motor is variable so that the hydraulic motor can be set in each case to the torque which is necessary to supply a secondary-side hydraulic actuator with pressure medium by means of the hydraulic pump. Hydraulic transformers can be embodied with various designs such as a radial piston machine, axial piston machine or vane machine.

WO 97/31 185 A1 discloses a hydraulic transformer with an axial piston design in which the hydraulic motor and hydraulic pump are integrated one in the other and which has a swash plate, a rotatably mounted drum with the axial pistons and a control cam with three kidney-shaped control slots whose relative position with respect to the dead center positions of the axial pistons can be varied by rotating the control cam with respect to the swash plate. Such a hydraulic transformer is extremely complicated to regulate.

The objective is to provide a hydraulic transformer in which complicated setting of the transmission ratio is avoided and which is simplified overall in terms of its control.

The aimed-at objective is achieved according to the invention by means of a hydraulic transformer which has the features of the preamble of patent claim 1 and in which in addition the control means can be controlled cyclically in accordance with the characterizing part of patent claim 1 and in which in particular a control cam or the expeller part can be driven in rotation by means of a drive, and in which of the two components comprising the expeller part and cam part one component can move freely with respect to the other component in terms of two rotational or translatory degrees of freedom within a limited range. According to patent claim 2, the control means can preferably be controlled cyclically, in particular one of the control cams can be driven in rotation by means of a drive, and of the two components comprising the expeller part and cam element one component is arranged essentially fixedly with respect to the housing and the other component can move freely in terms of two rotational or translatory degrees of freedom within a limited range.

Further advantageous embodiments of a hydraulic transformer according to the invention can be found in the further subclaims.

According to patent claim 3, the limits of the range within which the other component can move freely are variable. The hydraulic transformer can then be set to a large swept volume if the secondary-side hydraulic actuator is to be moved at high speed. At low speed, the swept volume is made small so that the control means can be operated with short cycle times and the pulsations in the streams of fluid are low. Static friction between the components which bear one against the other and are moved in relation to one another is less apparent than in the case of slow movements. The stream of fluid to the hydraulic actuator can be metered better.

According to patent claim 4, the cam element in a hydraulic transformer with an axial piston design is preferably a wobble plate which is mounted by means of a universal joint with its center in the center of said wobble plate so as to be capable of pivoting on all sides and can be supported, at a distance from its center, on a stop in a rotational fashion. The hydraulic transformer has a high dynamic level since the wobbling movement produces only low moments of inertia. On the one hand, in comparison with a hydraulic transformer with a rotatably mounted swash plate, the moved mass can be kept small, and on the other hand the moment of inertia of a circular disk about its central axis is twice as large as the moment of inertia with respect to an axis of symmetry in the disk plane. The axial forces of the drive mechanism can easily be absorbed hydrostatically since there is no need for a mechanical shaft bearing with seal.

The stop is advantageously steady in the direction of rotation of the wobble plate. This means that the support point or the support line of the wobble plate rotates steadily against the stop during operation and the wobble plate makes a steady and not a jolting wobbling movement with a respective slight change in the oblique setting.

The compressive load between the wobble plate and stop, and thus the wear and plastic deformation, are kept low by a linear abutment between the wobble plate and the stop.

According to patent claim 7, the distance between the center and the rotating support point of the wobble plate is equal to or larger than the distance between the center and the locations where the axial pistons act on the wobble plate. The contact force with respect to the force exerted by the axial pistons is then stepped down. If the distance is the same, the one dead center position during the movement of the axial pistons does not change when the oblique position of the wobble plate changes and the length of the bores in which the axial pistons are located can be very small.

In one hydraulic transformer according to patent claim 12, the distance between the universal joint and the stop measured in the direction of the central axis of the expeller part is variable. Given distances of different sizes, the oblique setting of the wobble plate, and thus the geometric swept volume of the hydraulic transformer, is different.

If the universal joint has a fixed position on a central axis of the hydraulic transformer, the rolling circle radius of the wobble plate on a support face is smaller than the rolling circle radius on the wobble plate. However, the rolling circle path on the support face is then shorter than on the wobble plate. When the wobble plate moves, compensation can then be carried out between the different lengths of the rolling circle paths by virtue of the fact that the wobble plate either also makes a rotational movement in addition to its wobbling movement or also slides with respect to the support face, at the rolling point. Sliding would mean increased wear at the punctual or linear contact point between the wobble plate and stop part. A rotational compensating movement of the wobble plate requires the expeller part to be rotatable about the central axis if the joints between the wobble plate and the axial pistons are fixed with respect to the wobble plate.

A compensating movement of the universal joint is preferably permitted. For this purpose, according to patent claim 13 the universal joint can be moved in the center of the wobble plate on a circular path about a central axis of the expeller part and the stop is of shell-shaped design in order to absorb axial and radial forces.

A further possibility is, according to patent claim 14, for the universal joint to be arranged fixedly on the central axis, and for a sliding element which bears against the stop in a plane perpendicular to the central axis and which is connected to the wobble plate by means of a joint whose position rotates with the wobble plate, to be arranged between the stop and the wobble plate. Of course, a planar sliding movement takes place between the sliding element and the stop. The wear which is caused by this is however low owing to the planar abutment between the wobble plate and stop.

A simple design for permitting the wobble plate to pivot on all sides and the oblique setting of the wobble plate to be varied is obtained if, according to patent claim 15, the wobble plate is embodied as a spherical layer which contains a large circle and which is located so as to slide in a sealed fashion in a circular-cylindrical receptacle and is supported in the direction of the expeller part, and if a hydraulic cushion, whose volume is variable, is located on the side of the wobble plate facing away from the expeller part.

According to patent claims 16 and 17, the diameter of a universal joint, which is embodied as a ball and socket joint, for the wobble plate can also made so large that the spherical bearing faces are located on the outside of the wobble plate, that is to say the wobble plate is in its entirety the positive part of the universal joint.

If, according to patent claim 18, the expeller part can be driven in rotation by means of a drive, the control cam can be arranged fixed to the housing so that the kidney-shaped control slots can be connected to the external ports without rotational connections.

Patent claims 19 to 21, contain advantageous embodiments of a hydraulic transformer according to the invention with a vane design, and patent claims 22 and 23 contain advantageous embodiments of a hydraulic transformer according to the invention with a radial piston design.

A plurality of exemplary embodiments of a hydraulic transformer according to the invention are explained in more detail below with reference to schematic drawings, in which:

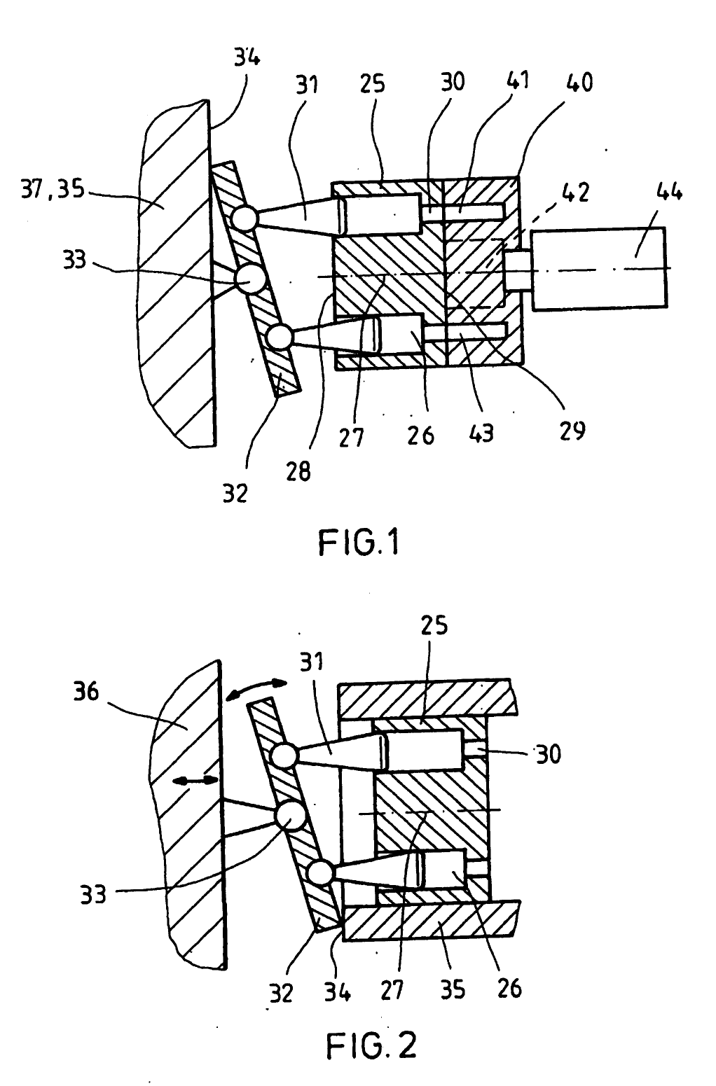

FIG. 1 shows an exemplary embodiment with an axial piston design, in which the expeller part which holds the axial pistons is fixed and a wobble plate is supported centrally by means of a fixed universal joint and is supported on its edge against a stop face,

FIG. 2 shows an exemplary embodiment with an axial piston design, in which the wobble plate is supported on its edge on the expeller part and the oblique setting of the wobble plate can be adjusted by displacing the universal joint.

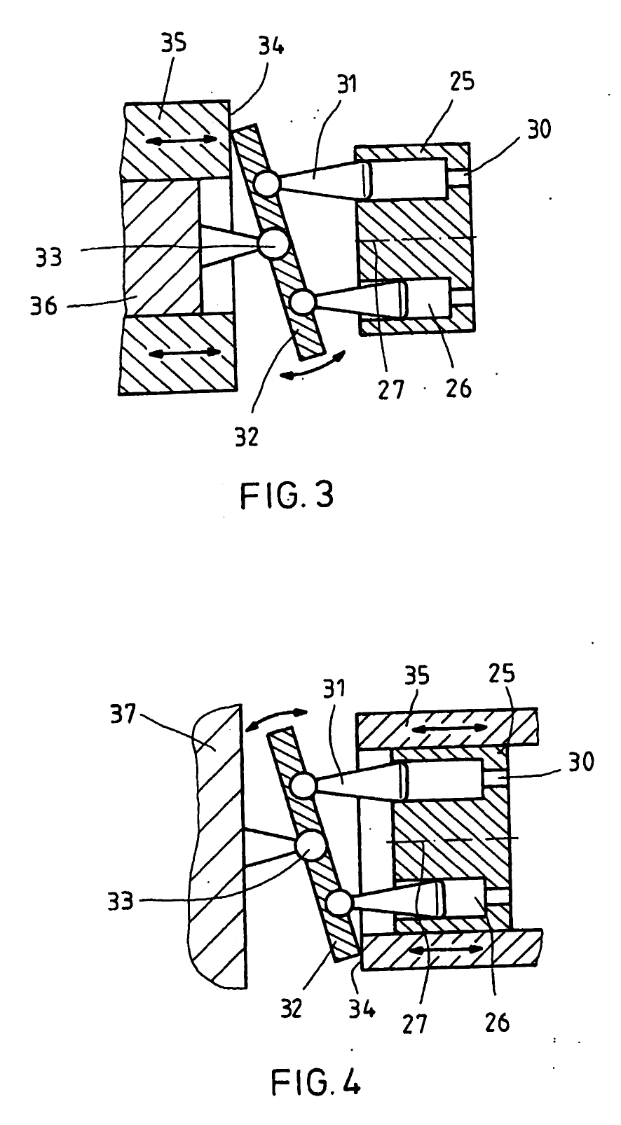

FIG. 3 shows an exemplary embodiment, similar to that from FIG. 1, in which however the oblique setting of the wobble plate can be adjusted,

FIG. 4 shows an exemplary embodiment, similar to that in FIG. 2, with the possibility of adjusting the oblique setting of the wobble plate by displacing the support point on the edge,

FIG. 5 shows an exemplary embodiment with an axial piston design with the possibility of adjusting the oblique setting of the wobble plate by adjusting the universal joint,

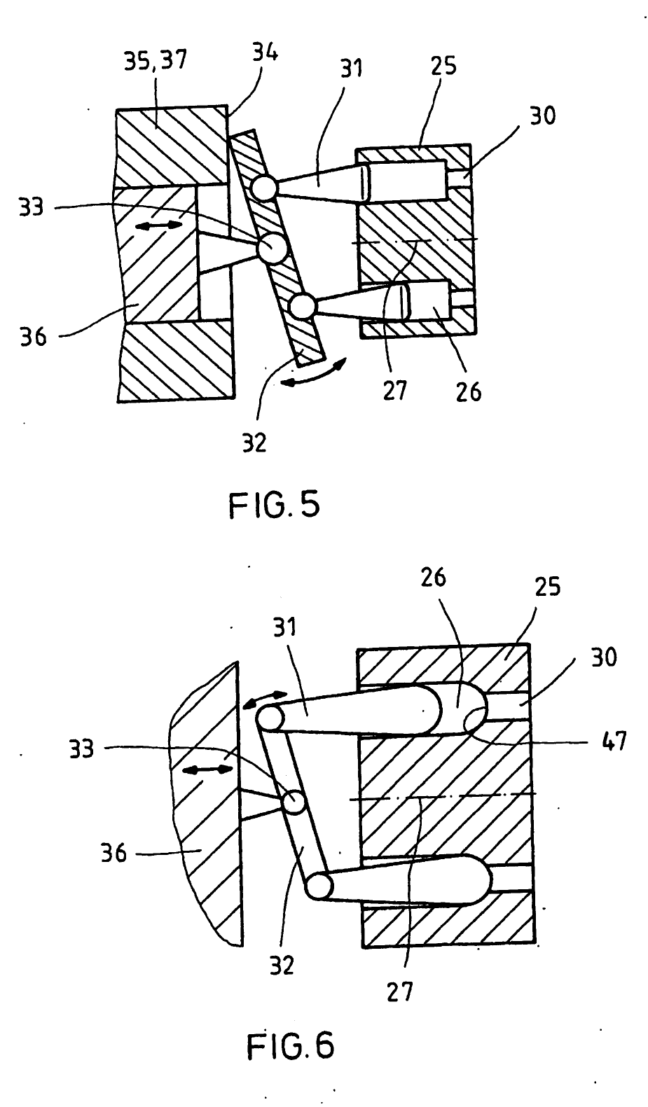

FIG. 6 shows an exemplary embodiment with an axial piston design, in which the wobble plate is supported by means of the axial pistons, and the oblique setting of the wobble plate can be adjusted by displacing the universal joint,

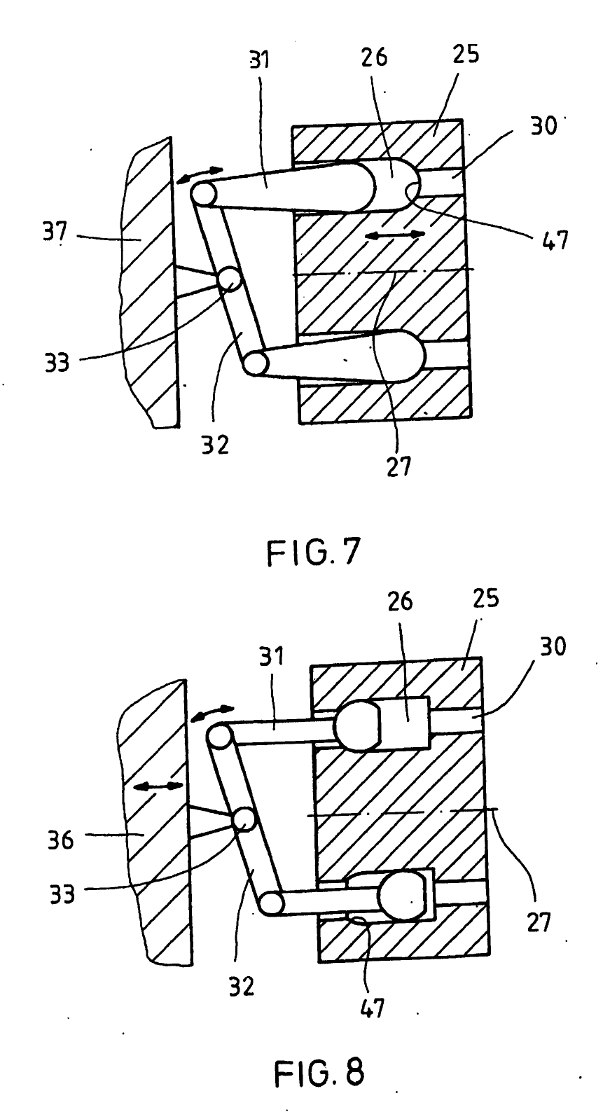

FIG. 7 shows an exemplary embodiment with an axial piston design, in which the wobble plate is supported by means of the axial pistons, and the oblique setting of the wobble plate can be adjusted by displacing the expeller part,

FIG. 8 shows an exemplary embodiment with an axial piston design, in which the wobble plate is supported by means of the axial pistons in the opposite direction to that in the sixth and seventh exemplary embodiments, and the oblique setting of the wobble plate can be adjusted by displacing the universal joint,

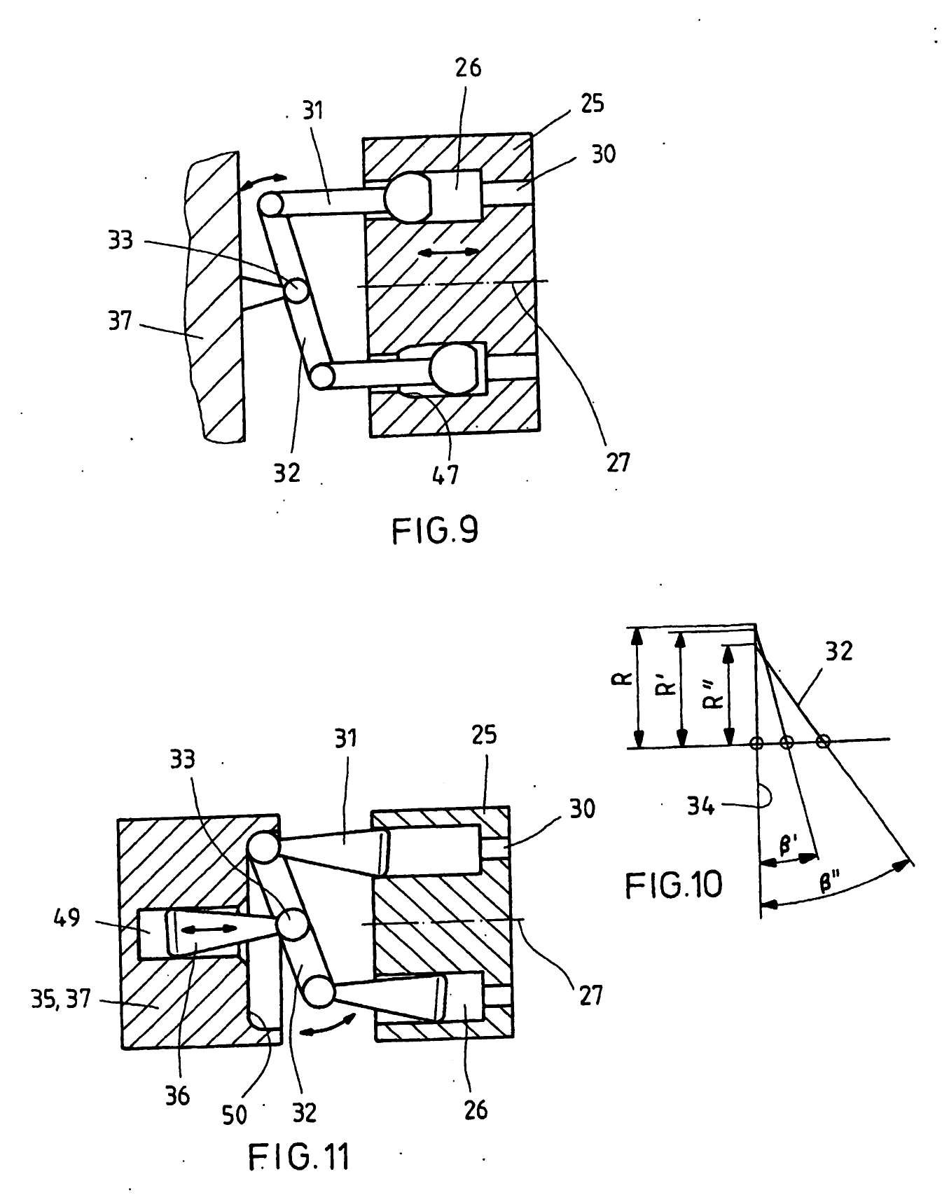

FIG. 9 shows an exemplary embodiment with an axial piston design, similar to that in FIG. 8, in which the oblique setting of the wobble plate can be adjusted by displacing the expeller part,

FIG. 10 shows the various supporting radii with various oblique settings of a wobble plate with a central universal joint which is arranged fixedly and perpendicularly with respect to the central axis,

FIG. 11 shows an exemplary embodiment with an axial piston design, in which the universal joint makes a compensating movement,

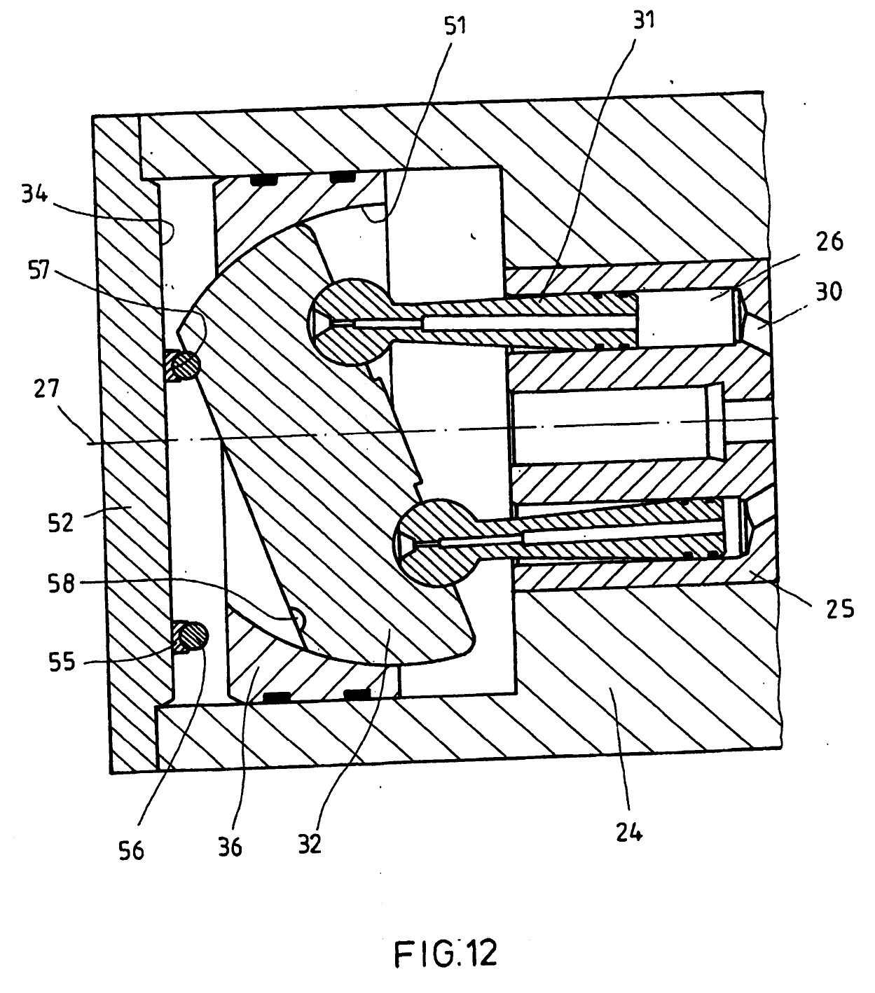

FIG. 12 shows an exemplary embodiment with an axial piston design, in which the wobble plate forms, in its entirety, the positive part of the displaceable universal joint and the wobble plate is supported on its edge by means of a supporting ring which can be displaced in a plane,

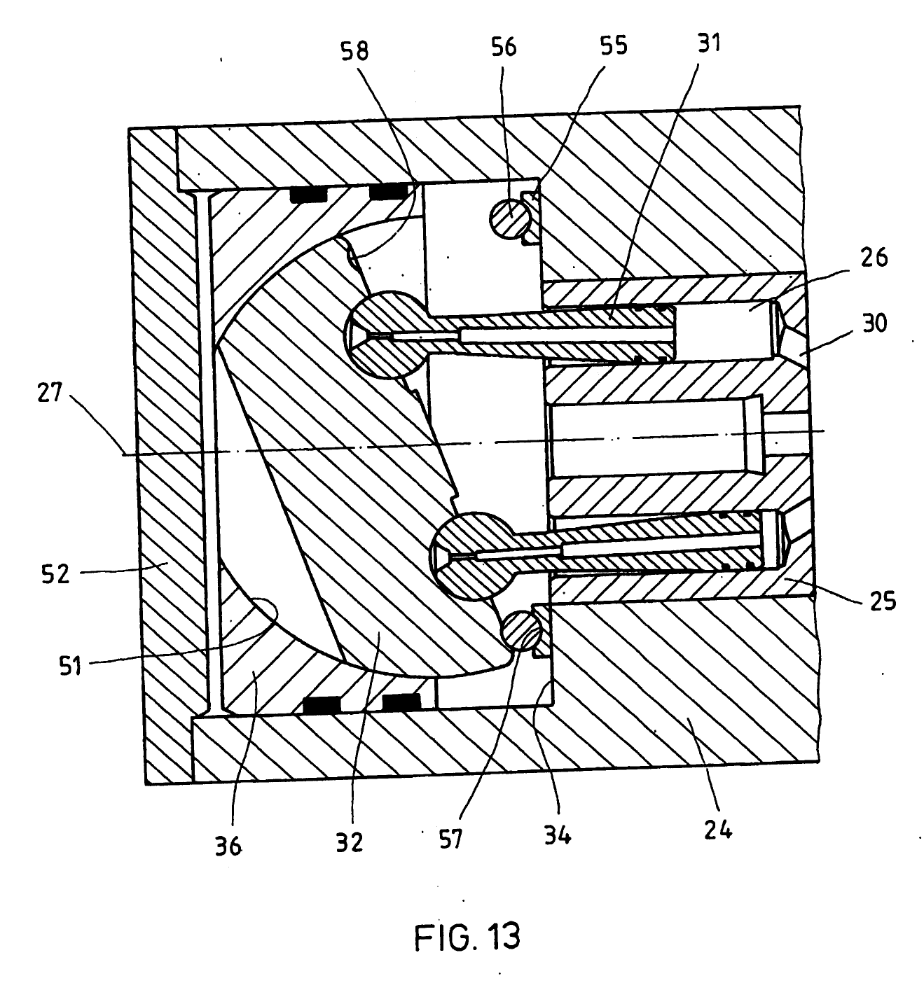

FIG. 13 shows an exemplary embodiment which is similar to the exemplary embodiment in FIG. 12, with the wobble plate being supported on the other side,

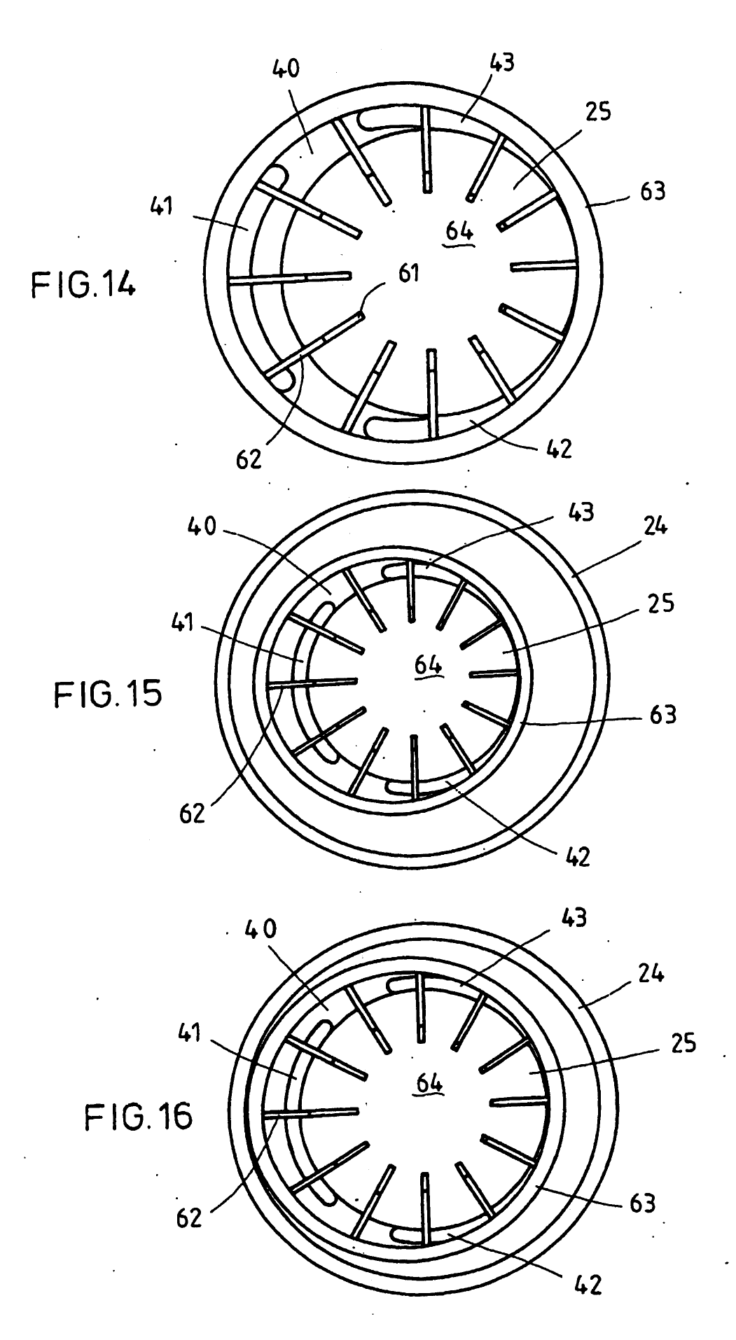

FIG. 14 shows an exemplary embodiment which is embodied with a vane design and in which the circular-cylindrical expeller part rolls freely on the inside of the housing,

FIG. 15 shows an exemplary embodiment which is also embodied with a vane design and in which a cam ring which surrounds the circular-cylindrical expeller part rolls freely on the outside of the expeller part,

FIG. 16 shows an exemplary embodiment which is also embodied with a vane design and in which a cam ring which surrounds the circular-cylindrical expeller part rolls freely on the inside of the housing,

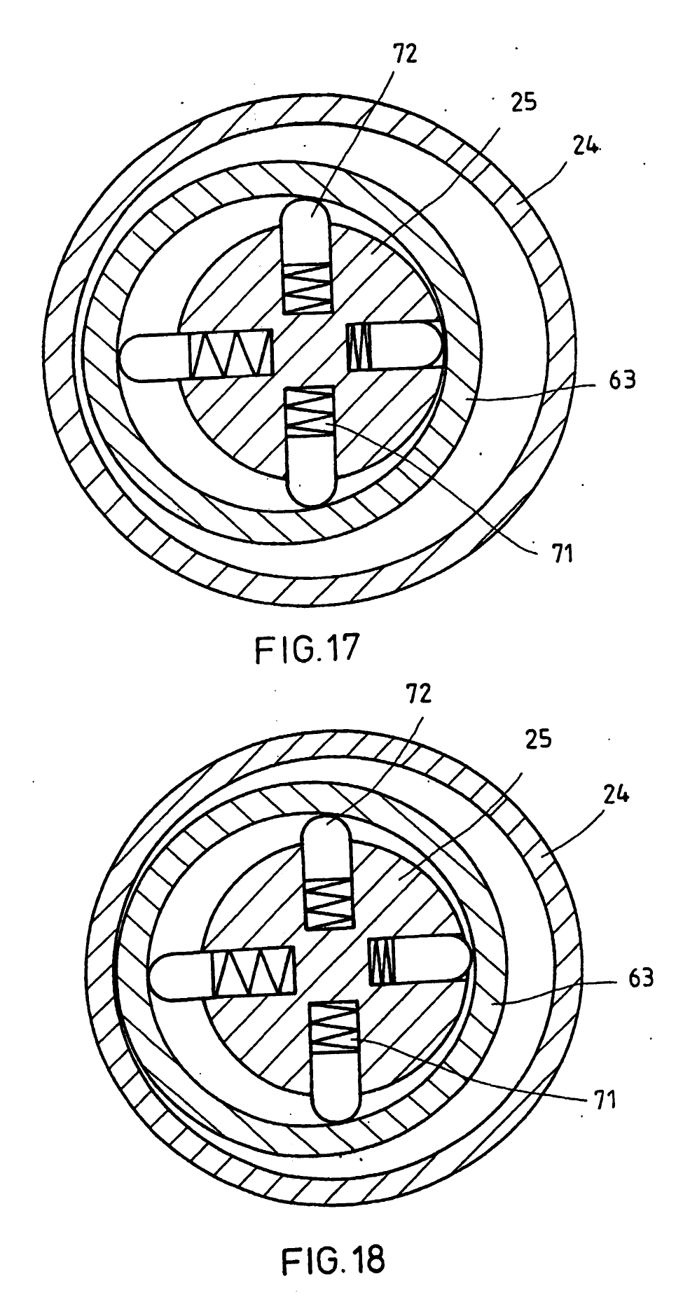

FIG. 17 shows an exemplary embodiment which is embodied with a radial piston design with radial pistons to which pressure is applied on the inside, and in which a cam ring which surrounds the circular-cylindrical expeller part rolls freely on the outside of the expeller part,

FIG. 18 shows an exemplary embodiment which is also embodied with a radial piston design with radial pistons to which pressure is applied on the inside, and in which a cam ring which surrounds the circular-cylindrical expeller part rolls freely on the inside of the housing,

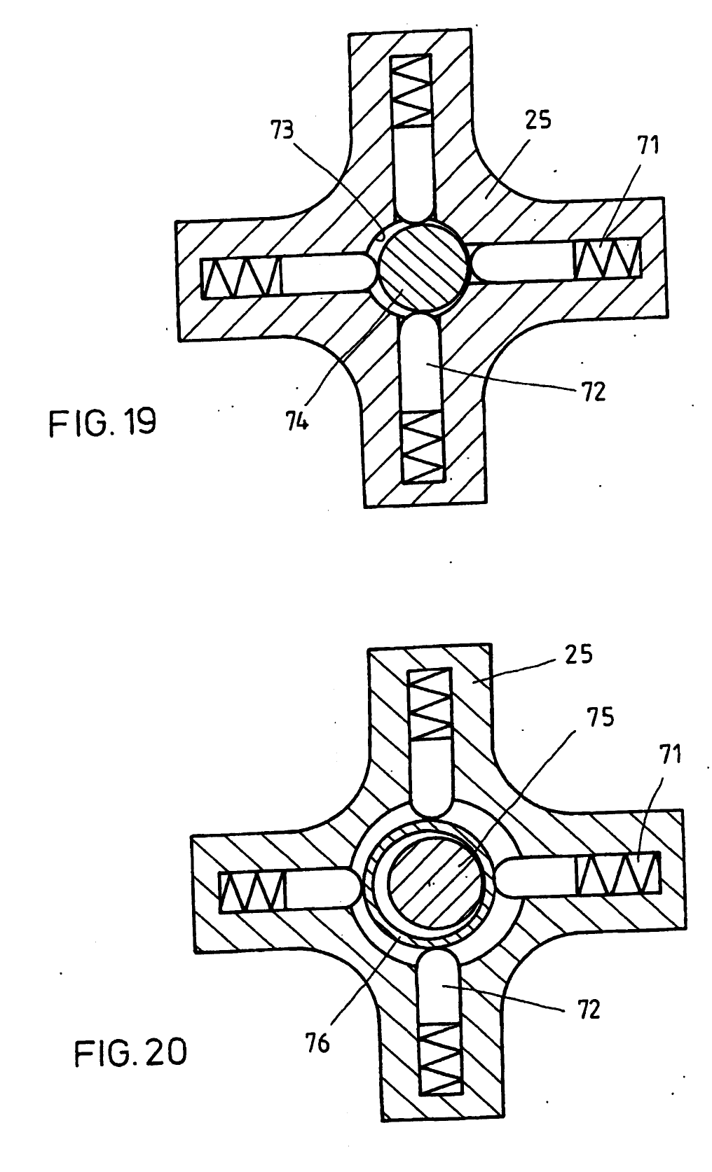

FIG. 19 shows an exemplary embodiment which is embodied with a radial piston design with radial pistons to which pressure is applied on the outside, and in which an eccentric disk rolls freely on the inside of the expeller part,

FIG. 20 shows an exemplary embodiment which is also embodied with a radial piston design with radial pistons to which pressure is applied on the outside, and in which an eccentric ring rolls freely on the outside on internal, fixed bolts, and

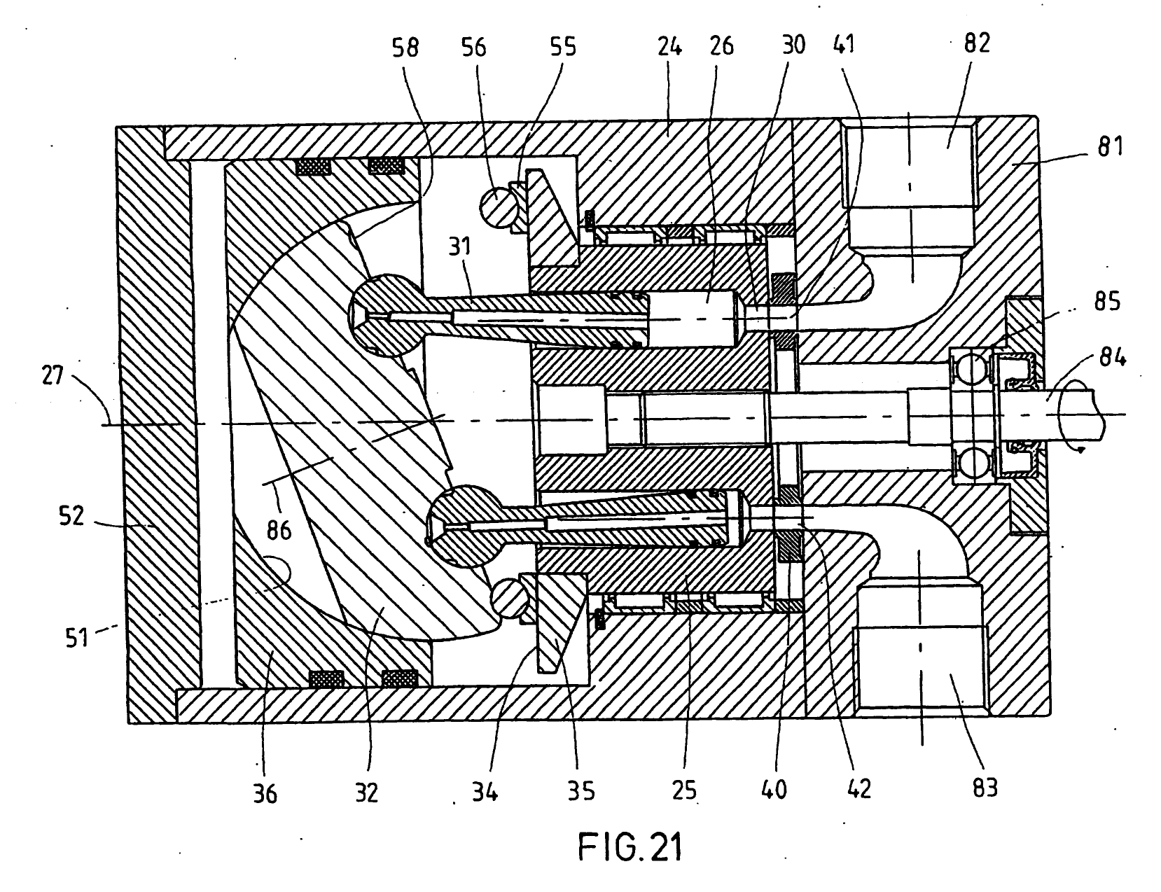

FIG. 21 shows an exemplary embodiment which is similar to that in FIG. 13 but in which it is not the control cam but rather the expeller part which can be driven in rotation.

According to the highly simplified section through the exemplary embodiment of a hydraulic transformer according to the invention in which is shown in FIG. 1, a fixed expeller part 15 has a plurality of cylinder bores 28 whose axes extend in parallel with one another, are at the same distance from a central axis 27 and whose angular spacings are identical to one another. The cylinder bores 26 are open on a first end side 28 of the expeller part. In each case a control bore 30 which is relatively small in diameter in comparison with the cylinder bore extends between the base of a cylinder bore and a second end side 29 of the expeller part. In each cylinder bore 26 there is a conical axial piston 31 whose conical head is coupled, so as to be capable of pivoting on all sides, to a wobble plate 32 which is located in front of the first end side 28 of the expeller part 25, in such a way that, on the one hand, the wobble plate can be pushed away from the axial piston by the expeller part, and on the other hand the axial piston does not lift off from the wobble plate. Overall, for example seven or ten axial pistons are provided.

The wobble plate 32 is a circular disk and is mounted so as to be pivotable on all sides by means of a fixed universal joint 33 whose pivot point or center lies in the center of the wobble plate and on the central axis 27. During operation, the wobble plate 32 lies, under the effect of the forces exerted on it by the axial pistons, with the edge of its side facing away from the expeller part 25 on a planar face 34—perpendicular to the central axis 27—of a positionally fixed stop part 35 which is part of a housing 37. The distance between the contact point on the face 34 and the center of the universal joint 33 is greater than the corresponding distance between the point where the resulting force acts on all the axial pistons so that the contact force is stepped down in comparison with the force exerted by the axial pistons. For a given size of the wobble plate 32, the distance between the center of the universal joint 33 and the face 34 determines the angular position or oblique setting of the wobble plate with respect to the central axis 27.

A control cam 40, in whose end side facing the expeller part 25 there are three kidney-shaped control slots, a kidney-shaped supply control slot 41, a kidney-shaped actuator control slot 42 and a kidney-shaped reservoir tank control slot 43, which are arranged on a circle, each extend over an angle of 90° and are at an angular spacing from one another of 30°, is located in a sealing manner at the second end side 29 of the expeller part. The distance between the kidney-shaped control slots and the central axis 27 is precisely as large as the distance between the control bores 30. The three kidney-shaped control slots are connected in a way which is not illustrated in more detail to a supply port which has the purpose of feeding fluid in from a constant pressure system and feeding fluid back into a constant pressure system, with an actuator port which has the purpose of feeding fluid to and feeding fluid back from a hydraulic actuator, and having a reservoir tank port which has the purpose of feeding fluid from and discharging fluid to a reservoir tank.

The control cam 40 can be driven in rotation about the central axis 27 by a rotational-speed-regulated electric motor 44 with a variable rotational speed.

If the electric motor 44 is switched off, the wobble plate 32 assumes a position which results from the sum of the forces exerted by the axial pistons to which the pressure of the constant pressure system and the load pressure of the hydraulic actuator are applied. If the control cam 40 is then made to rotate, the application of the pressure to the axial pistons migrates along with the kidney-shaped control slots of the control cam so that the wobble plate also changes the angular setting of its oblique setting and the contact point or the contact line on the face 34 rotates as is the case when a coin rotates with a wobbling motion on an underlying surface. Given a fixed oblique setting of the wobble plate and constant pressure conditions, the quantity of fluid which flows to the hydraulic actuator and flows back from it is determined here solely by the rotational speed of the control cam. If the supply pressure or the load pressure changes, this leads to a change in the relative angular position between a directional jet, defined by the center and the outer supporting point of the wobble plate, and the control cam.

The hydraulic transformer shown can thus very easily be controlled by the rotational speed of the control cam—and this applies generally to a hydraulic transformer according to the invention. Said hydraulic transformer is very operationally reliable since in the event of a fault, for example in the event of a break in an electrical cable, a fluid line or in the event of a loss of the supply pressure, the wobble plate moves into a specific obliquely angled position and remains there owing to the piston forces with their centering effect.

In the exemplary embodiment according to FIG. 2, the degree of the oblique setting of the wobble plate 32 is variable. The wobble plate is then supported with the edge of its front side, facing the expeller part 25, on the support face 34 of a stop part 35 which is fixed to the housing and which surrounds the expeller part 25. The universal joint 33 is located between the wobble plate and a joint carrier 36 which can be displaced with respect to the stop part 35, in the direction of the central axis 27. The axial distance between the center of the universal joint 33 and the supporting face 34, and thus the degree of the oblique setting of the wobble plate and the geometric swept volume of the hydraulic transformer, are therefore variable.

If the degree of the oblique setting of the wobble plate is variable, it is possible, on the one hand, to feed large quantities of pressure medium to the hydraulic actuator given a highly oblique setting, and on the other hand to control the actuator very accurately and with little pulsation given a low oblique setting of the wobble plate.

The exemplary embodiment according to FIG. 3 is equivalent to the first exemplary embodiment in terms of the control cam (not shown), in terms of the expeller part 25 and in terms of the wobble plate 32 with the universal joint 33 which is fixed to the housing. What is different is that the support face 34 for the edge of the wobble plate 32 is now located on an annular stop part 35 which can be displaced in the direction of the central axis 27 and which surrounds a carrier 36 of the universal joint. In the exemplary embodiment according to FIG. 3, the axial distance between the center of the universal joint 33 and the support face 34, and thus the degree of the oblique setting of the wobble plate and the geometric swept volume of the hydraulic transformer, can therefore be varied.

This is also the case in the exemplary embodiment according to FIG. 4. The universal joint 33 is located between the wobble plate and the positionally fixed housing part 37. The wobble plate is supported with the edge of its front side, which faces the expeller part 25, on the support face 34 of a stop part 35 which surrounds the fixed expeller part 25 and can be displaced in the direction of the central axis 27.

The exemplary embodiment according to FIG. 5 is of largely identical design to that according to FIG. 2 and has a carrier 36 for the universal joint 33 on the rear of the wobble plate 32, and an annular stop part 35, surrounding the carrier, with the support face 34. The stop part 35 is then fixedly arranged, and the carrier 36 can be displaced with the universal joint in the direction of the central axis 27.

In the exemplary embodiments according to FIGS. 6 to 9, the wobble plate 32 is not supported by a single planar face on its edge. Instead, end stops 47 for the axial pistons 31 in the cylinder bores 26 of the expeller part 25 serve as the stop for the wobble plate 32. In the two exemplary embodiments according to FIGS. 6 and 7, the end stops 47 are formed by the bottoms of the cylinder bores 26. These are rounded in a spherical shape. The internal ends of the axial pistons 31 are also correspondingly curved so that whenever the axial pistons are in an oblique setting a planar abutment of the axial pistons against the end stops 47 is ensured. In the exemplary embodiment according to FIG. 6, the expeller part 25 is arranged fixed to the housing, while the carrier 36 of the universal joint 33 can be displaced in the direction of the central axis 27. In the exemplary embodiment according to FIG. 7 it is reversed. The degree of the oblique setting of the wobble plate 32, and thus the swept volume of the respective exemplary embodiment, can therefore be adjusted. In the two exemplary embodiments according to FIGS. 8 and 9, the end stops 47 which are curved in a spherical shape are formed by mouths of the cylinder bores 26 which are reduced in diameter. Internal heads of the axial pistons 31 are correspondingly curved so that, here too, the planar abutment of the axial pistons against the end stops 47 is ensured for each oblique setting of the axial pistons. The axial pistons 31 are coupled to the wobble plate 32 by means of a joint which can be used to transmit not only compressive forces but also relatively large tensile forces from the axial pistons 31 to the wobble plate 32.

If the universal joint 33 in the exemplary embodiments according to FIGS. 1 to 5 has a fixed position on the central axis 27, the rolling circle radius of the wobble plate 32 on a support face 34 is smaller than the rolling circle radius on the wobble plate. The conditions are illustrated in FIG. 10. The rolling circle radius of the wobble plate is designated there by R. The wobble plate 32 therefore bears on the stop face 34 with points, measured in its plane, which are all at the same distance R from the center. In contrast, the rolling circle radius on the stop face 34 is smaller than R in every oblique setting of the wobble plate 32. Given an oblique setting with the angle β′, said radius is R′, and given an angle of β″, it is R″. However, if R′ and R″ are smaller than R, the rolling circle path on the support face 34 is shorter than on the wobble plate 32. When the wobble plate 32 moves, compensation is brought about between the different lengths of the rolling circle paths by the wobble plate either also making a rotational movement or sliding with respect to the support face, at the rolling point. In order to avoid sliding, the expeller part must be permitted to rotate about the central axis 27 if the joints between the wobble plate and the axial pistons are positionally fixed with respect to the wobble plate.

A further solution is that, as in the exemplary embodiment according to FIG. 11, a compensating movement of the universal joint 33 is permitted. One part of the universal joint is located on a pivotable hydraulic piston 48 which is supported by a fluid cushion in a cylinder bore 49 of the stop part 35 which is fixed to the housing. The stop part 35 has a shell-shaped recess 50 centrally with respect to the central axis 27, said recess 50 being bounded by a face which is perpendicular to the central axis, a circular-cylindrical edge with the central axis 27 as axis, and with a radius which is equal to the radius of the wobble plate 32, and a rounded portion with a specific radius between them. The edge of the wobble plate 32 has the same radius as the rounded portion of the recess 50 so that the wobble plate can fit snugly into the recess and the wobble plate bears against the stop part in a linear fashion.

As in the exemplary embodiments according to FIGS. 6 to 9, the articulation points of the axial pistons 31 bear against the wobble plate 32 also in the exemplary embodiment according to FIG. 11 at the same distance from the center of the wobble plate 32 as its external supporting edge. As a result of this, when the degree of the oblique setting of the wobble plate 32 changes, only one dead center changes in the movement of the axial pistons 31, while the other dead center is always the same. The length of the cylinder bores 26 then only has to be matched to the maximum cam of the axial pistons 31. The travel range always remains within the travel range for the maximum oblique setting. If the articulation points of the axial pistons are less far from the center of the wobble plate than the support edge, when the oblique setting of the wobble plate were reduced the travel region would move out of the travel range at the maximum oblique setting and the cylinder bores 26 would have to be longer.

During operation, the wobble plate 32 is loaded axially and radially by the axial pistons 31 which are located in the expeller part 25 which is arranged fixed to the housing, and is pressed into the rounded portion of the recess 50 independently of the degree of the pivoted position. There is therefore no rotational movement superimposed on the wobbling movement of the wobble plate. There is also no sliding between the wobble plate and the stop part. However, the center of the universal joint moves on a circular path about the central axis 27. The radius of the circular path is dependent on the pivot angle of the wobble plate. In the exemplary embodiment according to FIG. 11, this pivot angle can be changed by displacing the hydraulic piston 36 with respect to the stop 35 which is fixed to the housing, by supplying pressure medium to, or discharging it from, the cylinder bore 49.

In the exemplary embodiment according to FIG. 12, the axial pistons 31 are also held by an expeller part 25 which is arranged fixedly with respect to a housing 24. There are essentially two differences with respect to the exemplary embodiment according to FIG. 11. On the one hand, the spherical faces of the universal joint 33 which is embodied as a ball and socket joint are moved to the outside on the edge of the wobble plate 32. This is then a spherical layer which is located in a spherical shell 51 of the hydraulic piston 36 and the center point lies on the central axis 27. As a result of the hydraulic piston 36 and the wobble plate 32 the fluid cushion between these two parts and a housing lid 52 is separated by the space, connected to a leakage oil port, between the expeller part and an axial face 34 of the housing 24, on the one hand, and the hydraulic piston and wobble plate, on the other. The hydraulic piston can be displaced axially by changing the volume of the fluid cushion in order to change the oblique setting of the wobble plate.

The second essential difference lies in the method of compensation between the length of the rolling circle path of the wobble plate and the length of the rolling circle path on a supporting face with a fixed center of the universal joint on the central axis 27. A planar support ring 55 and a toroidal rolling ring 56, which lies in a groove 57—with a circular-segment-shaped cross section—of the supporting ring 55, are inserted between a planar support face 34 on the housing lid 52 and the wobble plate 32. The wobble plate 32 also has a circumferential groove 58 which is located centrally with respect to the axis of rotation of the wobble plate and whose cross section is a circular segment. The grooves 57 and 58 and the rolling ring 56 have the same diameter and the same curvature in cross section.

The wobble plate 32 bears in the groove 58 at a location on the rolling ring 56 in a linear fashion and via said rolling ring 56 presses the support ring 55 with a planar annular face against the support face 34 on the housing lid 52. When the wobble plate 32 wobbles, the linear contact point between the wobble plate and the rolling ring 56 moves along the latter in a pure rolling movement. The support ring 55 and rolling ring carry out a translatory compensating movement in a plane perpendicular to the central axis 27, while maintaining their orientation in the this plane, said movement having the same amplitudes in two directions perpendicular to one another. Owing to the planar abutment of the support ring 55 against the support face 34, hardly any wear occurs at the support ring or at the housing lid. The greater the oblique setting of the wobble plate, the greater the amplitude of the compensation movement of the support ring and rolling ring.

The exemplary embodiment according to FIG. 13 is largely the same as that according to FIG. 12. All that is different is that the support ring 55 and the rolling ring 56 are located in front of the side of wobble plate 32 which faces the expeller part 25. Correspondingly, said wobble plate 32 has the groove 58 on this side. The support ring is pressed by the wobble plate against a support face 34—located outside the expeller part 25—of the housing 24.

The hydraulic transformers with a vane design according to FIGS. 14 to 16 have a circular-cylindrical expeller part 25 which bears on a planar end side (concealed in the figures) on the rotatable control cam 40 which is provided with the three kidney-shaped control slots 41, 42 and 43, and holds vanes 62 as expellers in a series of radial slits 61 which are spaced apart from one another uniformly. The expeller part 25 is surrounded by a cam ring 63 which constitutes the cam part and whose internal diameter is larger than the external diameter of the expeller part 25.

In the exemplary embodiment according to FIG. 14, the cam ring 63 is arranged fixed to the housing and may be part of the housing. The kidney-shaped control slots 41, 42 and 43, which in turn extend over approximately 90° and have an angular spacing of 30°, are located along the internal contour of the cam ring 63. The expeller part 25 is a drum which also has an end side 64 which faces away from the control cam and extends perpendicularly to the axis of the cam ring 63. It can be moved freely within the cylindrical space, which is bounded radially by the cam ring 63 and axially by the control cam 40 and at the end side by the housing and whose axial extent is slightly greater between the blades 62, while ensuring end-side sealing of the chambers, than the axial extent of the expeller part 25, in a plane parallel to its end sides, that is to say in two directions which are perpendicular to one another. If, during operation, the control cam 40 is, for example, rotated by means of an electric motor 44, the expeller part 25 rolls on the inside on the cam ring 63, said expeller part 25 rolling once around the cam ring 63 during one revolution of the control cam given constant pressure conditions. Because the external circumference of the expeller part is smaller than the internal circumference of the cam ring, the expeller part also rotates by a specific angle about its own axis during one revolution. If the pressure conditions change, the assignment between the expeller part and the control cam also changes.

In the two exemplary embodiments according to FIGS. 15 and 16, the expeller part 25 with the vanes 62 is also arranged fixed to the housing. A cam ring 63, which can be moved in a plane perpendicular to the axis of the expeller part 25, is located in the housing 24, separated from it. The kidney-shaped control slots 41, 42, 43 then extend along the outer circumference of the expeller part 25 whose external circumference is in turn smaller than the internal circumference of the cam ring 63 which surrounds the expeller part.

During operation, the cam ring 63 rolls on the outside of the expeller part 25 in the exemplary embodiment according to FIG. 15, and rolls on a circular-cylindrical internal contour of the housing 24 in the exemplary embodiment according to FIG. 16.

The exemplary embodiment according to FIG. 17 of a hydraulic transformer with a radial piston design is very similar to the exemplary embodiment according to FIG. 15. Within a housing 24 there is a fixed, circular-cylindrical expeller part 25 which holds radial pistons 72 in radially extending bores 71, it being possible to apply pressure on the inside of said radial pistons 72. A cam ring 63 whose internal diameter is larger than the external diameter of the expeller part surrounds the expeller part and rolls on it during operation. The pressure spaces behind the radial pistons are connected in succession to a constant pressure system, a hydraulic actuator and a reservoir tank, for example using a control cam.

The exemplary embodiment according to FIG. 18 also has, in a housing 24, a fixed expeller part 25 with radial pistons 72, to which pressure is applied on the inside, and a freely movable cam ring 63. During operation, the latter then does not roll on the outside of the expeller part 25 but rather on the inside on a circular-cylindrical contour of the housing 24.

The two exemplary embodiments according to FIGS. 19 and 20 are constructed with a radial piston design with radial pistons 72 to which pressure is applied on the outside. Said radial pistons 72 are located in radial bores 71 in a fixed expeller part 25 which may be part of a housing. The radial pistons project into a central bore 73 in the expeller part 25, in which bore 73 a circular-cylindrical cam plate 74, whose diameter is smaller than the diameter of the bore 73, can move freely in the perpendicular direction with respect to the axis of the bore. During operation, the cam plate rolls on the internal contour of the bore 73.

Finally, in the exemplary embodiment according to FIG. 20, a fixed bolt 75, which is surrounded by a cam ring 76, is located centrally in the bore of the expeller part 25 which is enlarged in comparison with the exemplary embodiment according to FIG. 19. The radial pistons 72 bear against the outside of the cam ring. During operation, the cam ring 76 rolls on the outside of the bolt 75.

In the exemplary embodiments, only one control cam which can be driven in rotation and has three kidney-shaped control slots is shown as cyclically controlled control means. Even though it is complex it is nevertheless conceivable to replace such a control cam which rotates during operation by individual valves which can be actuated cyclically and by means of which the expeller spaces are successively connected to the pressure system, to the hydraulic actuator and to the reservoir tank.

In the exemplary embodiment according to FIG. 21, axial pistons 31 are held by an expeller part 25, as in the exemplary embodiment according to FIG. 12. However, the expeller part is rotatably mounted in a central part of the housing 24 and can be driven by means of a shaft 84 which passes through a connecting flange 81 with three outer ports, two ports 82 and 83 of which are shown, and a control cam 40, which is arranged fixedly with respect to the housing and has three kidney-shaped control slots, of which in each case two, for example the kidney-shaped control slots 41 and 42, are shown. The shaft is connected to the expeller part by means of a toothing so as to rotate with it and rotatably mounted in the connecting flange 81 by means of a roller bearing 85. The control cam could also be formed directly by the housing flange 81.

As in all the exemplary embodiments according to FIG. 1 to FIG. 13, the wobble plate 32 is mounted centrally by means of a universal joint 33. As in the exemplary embodiment according to FIG. 13, the spherical faces of the universal joint 33 which is embodied as a ball and socket joint is moved to the outside on the edge of the wobble plate 32. The latter is a spherical layer which is located in a spherical shell 51 of the hydraulic piston 36 whose center point lies on the central axis 27. By means of the hydraulic piston 36 and the wobble plate 32, the fluid cushion between these two parts and a housing lid 52 is separated from the space—connected to a leakage oil port—between the expeller part and an axial face 34 of the housing 24, on the one hand, and the hydraulic piston and wobble plate, on the other. The hydraulic piston can be displaced axially in order to change the oblique setting of the wobble plate.

The different length of the rolling circular path of the wobble plate 32 with respect to the length of the rolling circular path on a support face 34 is compensated, as in the exemplary embodiments according to FIGS. 12 and 13, by means of a support ring 55 and a toroidal rolling ring 56 which are located in front of the side of the wobble plate 32 which faces the expeller part 25. Correspondingly, said wobble plate 32 has, in the side in question, the groove 58 in which the bearing line of the rolling ring runs around on the wobble plate. The support ring 55 is pressed by the wobble plate 32 against a support face 34 on a stop plate 35 which is located on the expeller part 25 and rotates with the expeller part.

If the expeller part 25 is driven during operation, the cylindrical bores 26 successively enter into fluidic connection with the kidney-shaped control slots 41 to 43 via the control bores 30. The wobble plate 32 is also made to rotate by means of the axial pistons 31 or a driver device (not illustrated). Since the position of the kidney-shaped control slots is fixed with respect to the housing 24, the oblique setting of the wobble plate remains fixed with respect to the housing during the movement as long as the pressure conditions do not change. The wobble plate therefore rotates about its axis 86 which runs obliquely with respect to the central axis 27. However, in relation to the expeller part 25, the wobble plate 32 carries out a wobbling movement during which the linear contact point between the wobble plate and the rolling ring 56 migrates along the latter in a pure rolling movement. The support ring 55 and rolling ring carry out a translatory compensating movement in a plane perpendicular to the central axis 27, while maintaining its orientation in this plane, said movement having the same amplitudes in two directions which are perpendicular to one another.

1-23. (canceled)

24. A hydraulic transformer comprising: a housing (24) and having an expeller part (25) in which a plurality of expellers (31, 62, 72) which bound expeller spaces with variable volumes are guided, a cam part (32, 63, 74, 76) on which the expellers are supported, and having control means comprising a control cam (40) which has three kidney-shaped control slots (41, 42, 43) via which the expeller spaces (26) can be successively connected to a supply port, to a working port and to a reservoir tank port, wherein the control means can be controlled cyclically, in that a control cam (40) or the expeller part (20) can be driven in rotation by means of a drive (44), and in that of the two components comprising the expeller part (25) and cam part (32, 63, 74, 76), one component can move freely with respect to the other component in terms of two rotational or translatory degrees of freedom within a limited range.

25. The hydraulic transformer as claimed in claim 24, wherein the control means can be controlled cyclically, in that a control cam (40) can be driven in rotation by means of a drive (44), and in that of the two components comprising the expeller part (25) and cam part (32, 63, 74, 76), one component is arranged essentially fixedly with respect to the housing (24) and the other component can move freely in terms of two rotational or translatory degrees of freedom within a limited range.

26. The hydraulic transformer as claimed in claim 24, wherein the limits of the range within which the other component can move freely are variable.

27. The hydraulic transformer as claimed in claim 24, further comprising axial pistons (31) which are located in the expeller part (25), and wherein the cam part (32) is a wobble plate which is mounted by means of a universal joint (33) with its center in the center of said wobble plate so as to be capable of pivoting on all sides and can be supported, at a distance from its center, on a stop (35) in a rotational fashion.

28. The hydraulic transformer as claimed in claim 27, wherein the stop (35) is steady in the direction of rotation of the wobble plate (32).

29. The hydraulic transformer as claimed in claim 28, wherein an abutment between the wobble plate (32) and the stop (35) is linear.

30. The hydraulic transformer as claimed in claim 28, wherein the distance between the center and the rotating support point of the wobble plate (32) is equal to or larger than the distance between the center and the locations where the axial pistons (31) act on the wobble plate (32).

31. The hydraulic transformer as claimed in claim 27, wherein the stop (35) for the rotating support point of the wobble plate (32) is located on its rear side facing away from the axial pistons (31).

32. The hydraulic transformer as claimed in claim 27, wherein the stop (35) for the rotating support point of the wobble plate (32) is located on its front side facing the axial piston (31).

33. The hydraulic transformer as claimed in claim 27, wherein the stop for the wobble plate (32) is implemented by means of a travel movement limitation for the axial pistons (31).

34. The hydraulic transformer as claimed in claim 33, wherein the axial pistons (31) and the bores (26) of the expeller part (25) in which the axial pistons (31) are located have faces (47) which correspond to one another, curve in a spherical or circular-cylindrical fashion, lie axially opposite one another and go into an abutting position against one another.

35. The hydraulic transformer as claimed in claim 27, wherein the distance between the universal joint (33) and the stop (35) measured in the direction of the central axis (27) of the expeller part (25) is variable.

36. The hydraulic transformer as claimed in claim 35, wherein the universal joint (33) can be moved in the center of the wobble plate (32) on a circular path about a central axis (27) of the expeller part (25), and in that the stop (35) is of shell-shaped design in order to absorb axial and radial forces.

37. The hydraulic transformer as claimed in claim 35, wherein the universal joint (33) is arranged fixedly on the central axis (27), and in that a sliding element (55) which bears against the stop (35) in a plane perpendicular to the central axis (27), and which is coupled to the wobble plate (32) by means of a joint whose position rotates with the wobble plate (32), is arranged between the stop (35) and the wobble plate (32).

38. The hydraulic transformer as claimed in claim 35, whereub the wobble plate (32) comprises a spherical layer which contains a large circle and which is located so as to slide in a sealed fashion in a circular-cylindrical receptacle, and is supported in a of the expeller part (25), and wherein a hydraulic cushion, whose volume is variable, is located on the side of the wobble plate (32) facing away from the expeller part (25).

39. The hydraulic transformer as claimed in claim 27, wherein the universal joint (33) is a ball and socket joint, and wherein the wobble plate (32) is embodied as a spherical layer with an outer face which lies on a spherical surface, and is held in a recess (51) with a spherical bearing face.

40. The hydraulic transformer as claimed in claim 39, wherein the recess (51) has a negative spherical layer, and in that the wobble plate (32) is supported on the stop (35) on a side facing away from the expeller part (25).

41. The hydraulic transformer as claimed in claim 27, wherein the expeller part (25) can be driven in rotation by means of a drive (44), and wherein the wobble plate (32) can be entrained by the expeller part (25), by means of the axial pistons (31).

42. The hydraulic transformer as claimed in claim 24, further comprising a vane design with a cam ring as cam part (63), having vanes (62) as expellers and having an expeller part (25) which holds the vanes, and wherein, of the two components comprising the cam ring (63) and expeller part (25), one component is arranged fixedly and the other component is supported radially on the inside or outside of a circular-cylindrical face, so as to be movable in a plane perpendicular to the axis of the fixed arranged component.

43. The hydraulic transformer as claimed in claim 42, wherein the expeller part (25) can be moved within a circular-cylindrical chamber of the housing (24) which functions as a cam ring (63).

44. The hydraulic transformer as claimed in claim 42, wherein the expeller part (25) is the fixedly arranged component, and the cam ring (63) is supported in a movable fashion within the housing (25) and on the inside of the expeller part (25) or on the outside of the housing (24).

45. The hydraulic transformer as claimed in claim 24, further comprising a radial piston design with a cam ring (63) as the cam part, with radial pistons (72) to which pressure is applied on the inside, as expellers, and with a fixed expeller part (25) which holds the radial pistons (72), and wherein the cam ring (63) can move within the housing (24) and is supported on the inside of the expeller part (25) or on the outside of the housing (24).

46. The hydraulic transformer as claimed in claim 24, further comprising a radial piston design with a cam ring (76) or a cam plate (74) as the cam part, with radial pistons (72) to which pressure is applied on the outside, as expellers, and with a fixed expeller part (25) which holds the radial pistons (72), and in that the cam part (74, 76) can move within the expeller part (25) and is supported on the inside or outside as a cam ring (76), and is supported on the outside as a cam plate (74).
